(19) United States
(12) Patent Application Publication
(10) Pub. No.: US 2011/0301505 A1
(43) Pub. Date: Dec. 8, 2011

(54) Methods, Devices, and Systems for Health Management

(76) Inventors: Shiow-Chen CHEN WANG, Taipei (TW); Hen-Te Liao, Taipei (TW)

(21) Appl. No.: 13/152,295

(22) Filed: Jun. 3, 2011

Related U.S. Application Data
(60) Provisional application No. 61/351,285, filed on Jun. 3, 2010.

(51) Int. Cl. A61B 5/11; G06Q 50/00; A61B 5/00

(52) U.S. Cl. 600/595; 600/300; 705/2

(57) ABSTRACT

The present invention discloses a portable health management device. The device includes a measuring element measuring a biological measurement for a user; a storage element storing a plurality of experience data associated with the biological measurement; an analysis module analyzing the biological measurement in real time based on the plurality of experience data so as to generate an analysis result, wherein the analysis result provides the user with a suggestion to improve the biological measurement so as to manage the user's health.

This application is related to and claims priority to U.S. provisional patent application, U.S. Provisional Application No. 61/351,285, filed on Jun. 3, 2010, by the applicants Hen-Te Liao and Shiow-Chen Chen Wang, entitled “Methods, Devices, and Systems for Health Management.”

### FIELD OF THE INVENTION

The present invention relates to methods, devices, and systems for health management; in particular, the present invention relates to methods, devices and systems capable of analyzing a user's short term, mid term, and long term biological measurements so as to provide the user with suggestions to improve his or her biological measurements, thereby managing the user's health.

### BACKGROUND OF THE INVENTION

People have focused more and more on their health management in recent years. However, the biological measuring devices available on the market can only provide simple measuring functions with no real time analyzing capabilities. The conventional biological measuring devices also cannot provide any suggestion to the user as to how to improve his or her health condition based on the biological measurement obtained. As a result, the user cannot immediately change his or her living habits that may be affecting his or her biological measurement. Instead, the user relies on doctors or professional medical staff for advice and reminder on how to improve his or her health condition.

For example, the conventional weight scales merely provide weight measurements. A few of the conventional weight scales further provide body fat readings. However, the conventional weight scales do not provide the user with any suggestion on how to improve the weight and/or body fat measured. For example, after an overweight user realizes he or she has a weight gain, the user might want to know how to reduce his or her weight effectively. Or, a user who finds out his or her body fat is too high may want to know how to burn body fat effectively. However, the conventional biological measuring devices may only give out readings related to the user's health condition without providing the user with real time suggestion on how to improve those readings based on the user's health condition.

In another example, the conventional blood sugar measuring devices merely show the blood sugar value measured from a tester. However, with respect to a tester with a biological condition different from the general public, the blood sugar value measured may be evaluated differently. Unfortunately, the conventional art does not analyze the biological measurement in real time with respect to a particular biological condition, let alone providing a suggestion to improve the biological measurement obtained.

Furthermore, the conventional biological measuring devices do not have enough internal memory to store a large amount of data. In other words, the conventional biological measuring devices may not be able to store the user's biological measurement after each take, or may only be able to store a few biological measurements, for example up to five measurements. As such, the user cannot maintain his or her long term health data in the conventional biological measuring devices, preventing the user to track his or her long term health trend based on the biological measurements obtained.

Therefore, what is needed is a biological measuring device capable of providing the user with a suggestion to improve his or her health condition. Preferably, the biological measuring device can conduct real time analysis on the biological measurement obtained based on experience data associated with the biological measurement so as to generate an analysis result and provide the user with real time suggestion to improve his or her health condition.

Additionally, what is needed in the art is a biological measuring device capable of tracking the user's long term health. Furthermore, the ideal device may merge the user's long term biological measurements into integrated health data so that the integrated health data may be analyzed, thereby showing the user's long term health trend.

### SUMMARY OF THE INVENTION

In light of the drawbacks of the current art, one aspect of the present invention is to provide a portable health management device. The device of the present invention may include a measuring element measuring a biological measurement for a user; a storage element storing a plurality of experience data associated with the biological measurement; an analysis module analyzing said biological measurement in real time based on the plurality of experience data so as to generate an analysis result, wherein the analysis result provides a suggestion to the user in real time to improve the biological measurement so as to manage the user's health.

Another aspect of the present invention is to provide a health management terminal device. The terminal device of the present invention may include a measuring unit measuring a biological measurement for a user; a storage unit storing at least one monitoring condition associated with the biological measurement; a health management module analyzing the biological measurement in real time based on the at least one monitoring condition so as to generate a professional report, wherein the professional report provides a suggestion to the user in real time to improve the biological measurement so as to manage the user's health.

Yet another aspect of the present invention is to provide a health management system. The system of the present invention may include a server. The server may include a registration mechanism establishing an account for a user to enter the system; a database storing an integrated health data associated with the user; an analyzing mechanism analyzing the integrated health data in real time to generate an integrated health report, wherein the integrated health data integrates a plurality of biological measurements transmitted to the server from another device, and wherein the user uses the account to enter the system and accesses the integrated health data, the integrated health report or a combination thereof associated with the user.

Yet another aspect of the present invention is to provide a health management system. The system of the present invention may include a health management user-end device. The device may include a measuring element measuring a biological measurement for a user; a network communication interface allowing the health management user-end device to upload a plurality of biological measurements to a server. The server may include an analyzing mechanism analyzing the plurality of biological measurements in real time to generate an integrated health report; and a user interface allowing the user to access the integrated health report.

Yet another aspect of the present invention is to provide a method for health management. The method of the present invention may include measuring a biological measurement from a user end of a health management system; uploading the biological measurement to a remote end of the health management system through a network; integrating a plurality of biological measurements into integrated health data from the remote end of the health management system; and analyzing the integrated health data in real time to generate an integrated health report.

### BRIEF DESCRIPTION OF DRAWING

FIG. 1 illustrates a block diagram of an exemplary portable health management device in accordance with one embodiment of the present invention;

FIG. 2 illustrates a block diagram of an exemplary health management terminal device in accordance with one embodiment of the present invention;

FIG. 3 illustrates a block diagram of an exemplary health management system in accordance with one embodiment of the present invention; and

FIG. 4 illustrates a flow chart of an exemplary method for health management in accordance with one embodiment of the present invention.

### DETAILED DESCRIPTION OF THE INVENTION

Reference will now be made in detail to the embodiments of the present invention. Examples of embodiments are illustrated in the accompanying drawings, wherein like reference numbers refer to like elements throughout the specification.

The present invention discloses methods, devices and systems for health management. In accordance with one embodiment of the present invention, the device may measure a user's biological measurement and analyze the biological measurement based on a plurality of experience data associated therewith in real time to generate an analysis result, wherein the analysis result may provide the user with a suggestion to improve the biological measurement in real time, thereby managing the user's health.

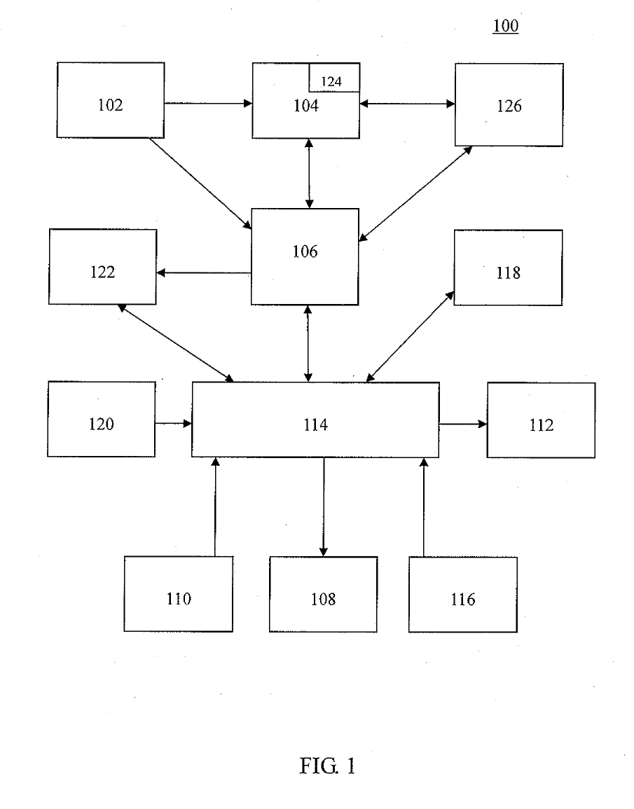

Refer to FIG. 1. FIG. 1 illustrates a block diagram of an exemplary portable health management device in accordance with one embodiment of the present invention. The portable health management device of the present invention may be administered by an individual user to monitor the user's health, for example, on a daily basis. For example, the portable health management device of the present invention may be a blood sugar measuring device, a blood pressure measuring device, a thermometer, a body fat measuring device, or any other similar devices capable of obtaining a biological measurement for a user. As shown in FIG. 1, the portable health management device 100 of the present invention may include a measuring element 102 capable of measuring a user's biological measurement. For example, the measuring element 102 may measure a user's blood pressure, blood sugar, body temperature, weight, body fat, heart beat or any other biological measurements.

In accordance with the present invention, the portable health management device 100 may include a storage element 104. In accordance with one embodiment of the present invention, the storage element 104 may store the biological measurements measured by the measuring element 102, such as the user's blood pressure, blood sugar, body temperature, weight, body fat and/or heart beat. In accordance with another embodiment of the present invention, the storage element 104 may also store a plurality of experience data associated with the above-mentioned biological measurements. In accordance with one embodiment of the present invention, the experience data may be all information and knowledge about historical events and processes observed from long term clinical care that can be described by data or structured qualitative descriptions based on the characteristics of the biological measurement. For example, with respect to the characteristics of blood sugar, because blood sugar levels change before and after food intake, the associated experience data may be the variation of the blood sugar levels before and after each meal of a patience monitored on a long-term basis. Take another example of the characteristics of blood pressure. Because blood pressure may change depending on a certain time during a day and/or an activity involved, the associated experience data may be the variation of the blood pressures measured in the morning and in the afternoon of a patient with high blood pressure. Take yet another example of the characteristics of body temperature. Because a pregnant woman usually has a higher body temperature, the associated experience data may be the average temperature of a woman with a baby in normal health condition. In accordance with a preferred embodiment of the present invention, the experience data combines the professional knowledge of patient care attendants and the long term clinical care data. In other words, the experience data can reflect the variation of different biological measurements due to the difference in each individual's physical health condition, age and other parameters.

In one embodiment of the present invention, the storage element 104 may further include a removable memory capable of providing additional storage to store a large amount of biological measurements, such as short-term, mid-term and long-term measurements depending on the capacity of the storage space. In another embodiment of the present invention, the storage element 104 may be a memory installed within the chipset of the portable health management device 100 to provide high speed computation, direct access, data security and stability. Because the biological measurements stored in the storage element 104 may be personal data involving privacy and/or security issues, in one embodiment of the present invention, the storage element 104 may also include an encryption mechanism 124 to perform encryption on data stored therein.

The portable health management device 100 of the present invention may include an analysis module 106. In accordance with one embodiment of the present invention, the analysis module 106 may perform real time analysis on the biological measurement measured by the measuring element 102 based on the plurality of experience data stored in the storage element 104 and generate an analysis result. Because the experience data utilized by the analysis module 106 to conduct the analysis factors in variation in measurements caused by a particular physical condition of an individual, the analysis result generated by the analysis module 106 may provide a more precise decision on the individual's health condition based on the characteristics of the biological measurement of a particular body type. For example, when performing an analysis on the body temperature of a pregnant woman, the analysis module 106 will consider the fact that the body temperature of a pregnant woman is generally higher than non-pregnant women to determine whether the body temperature measured by the measuring element 102 is normal.

In one embodiment of the present invention, the analysis result may provide the user with a suggestion to improve the biological measurement so as to manage the user's health. For example, if the blood pressure measured for a user with a history of high blood pressure is higher than the average measurements obtained in recent days, the analysis result may indicate that the user's blood pressure is not normal and suggest the user to take medication, relax, or pay attention to the food intake. As such, the user may improve his or her blood pressure without seeing a doctor or medical staff. In one embodiment of the present invention, the storage element 104 may store the analysis result generated by the analysis module 106 for the user's future reference.

In accordance with one embodiment of the present invention, the analysis module 106 may further perform analysis on a plurality of biological measurements stored in the storage element 104 so as to generate a more precise analysis result. The difference between performing a real time analysis on a single biological measurement and with a plurality of biological measurements is that the availability of the plurality of biological measurements allows the analysis module 106 to perform minor adjustment to the analysis result so that a more appropriate suggestion may be given to the user based on his or her particular physical condition. In accordance with one embodiment of the present invention, the analysis result may be shown to the user in text, graphics or the combination thereof to help the user understand the content of the analysis result.

The portable health management device 100 of the present invention may include an output element 108. In accordance with one embodiment of the present invention, the output element 108 may include but not limited to a display screen or a speaker installed on the portable health management device 100, an external printer, a portable drive, or any other device capable of outputting the analysis result. In accordance with one embodiment of the present invention, the output element 108 may output the analysis result in voice, graphics, text, data file, or any combination thereof.

The portable of health management device 100 of the present invention may include an input element 110. In accordance with one embodiment of the present invention, the input element 110 may include by not limited to a mouse, keyboard, speaker stalled on the portable health management device 100 or other external input elements. In accordance with one embodiment of the present invention, the input element 110 may allow the user to enter a target value to be used as a basis for the analysis module 106 to analyze the biological measurement. For example, the user can enter a target weight. Whenever the analysis module 106 performs an analysis on the weight measured by the measuring element 102, the target weight may be added as one of the basis for the analysis. If the weight measured for an overweight user is higher than the target value, then the analysis result generated by the analysis module 106 can provide the user with suggestions on how to reduce his or her weight. If the weight measured for an underweight user is lower than the target value, then the analysis result generated by the analysis module 106 can provide the user with suggestions on how to increase his or her weight. In one embodiment of the present invention, the storage element 104 may also store the target value entered by the user for future use.

The portable health management device 100 of the present invention may a scheduling module 122. The scheduling module 122 may put the suggestion provided in the analysis result into schedule to regularly remind the user to execute the suggestion. For example, for a user who wants to lose weight, if the analysis result provided by the analysis module 106 shows the user's weight is higher than the target value, then the analysis result can suggest the user to exercise one hour daily. As such, the scheduling module 122 can put an exercise activity into the user's schedule, for example, a reminder may be sent to the user to work out for an hour at 7 pm everyday.

The portable health management device 100 of the present invention may include a warning element 112. The warning element 112 of the present invention may selectively send out a warning signal based on the analysis result. For example, if the analysis result shows the user's blood pressure exceeds an expected value, then a warning light installed on the portable health management device 100 can flash to warn the user. In one embodiment of the present invention, the warning light can indicate different levels of warning by colors. For example, a green light can indicate the analysis result is normal, whereas a red light can indicate the analysis result shows an abnormal biological measurement, etc. In another embodiment of the present invention, the warning element 112, such as a warning light, can also flash or remain still to remind the user of his or her health condition. For example, the flashing light can indicate the analysis result shows an abnormal biological measurement, whereas a still light can indicate the analysis result is normal. However, it would be apparent to those skilled in the art that warning element 112 can be embodied in ways other than a color or flashing light. The warning element 112 can also be implemented in voice, vibration or any other way to remind the user of his or her health condition.

The portable health management device 100 of the present invention may include a user interface 114 so that the user can control the portable health management device 100, for example, by voice or graphics. In one embodiment of the present invention, the user interface 114 may utilize a graphic icon or text message to guide the user to control the portable health management device 100.

The portable health management device 100 of the present invention may include a force detection module 116. The force detection module 116 may control the user interface 114 by a direction of force. For example, if the user wishes to return to the previous content shown on the user interface 114, the can tilt the portable health management device 100 to the left or to the right to change the content shown on the user interface 114. In one embodiment of the present invention, the force detection module 116 may ask the user to perform a certain movement and determine the user's health condition based on a characteristic of the movement. For example, the force detection module 116 of the present invention may request the user to move the portable health management device 100 up and down so as to determine the user's physical and/or mental condition through the strength of the movement. For example, if the user has trouble raising the portable health management device 100 up after he or she is asked to do so, the force detection module 116 may determine the user's low blood pressure has made him or her too weak to move, and thus help is required.

In accordance with the present invention, the portable health management device 100 may include a network communication module 118. The network communication module 118 may allow communication with an external device or with another person through the network. For example, the user may upload the biological measurement and/or analysis result stored in the storage element 104 to an external device. In another example, the user may conduct a video conference with another person using the network communication module 118.

The portable health management device 100 of the present invention may further include a data transmission interface 126. In one embodiment of the present invention, the data transmission interface 126 may transmit the biological measurement to an external device or receive the biological measurement from the external device using an RS 232 or USB interface.

The portable health management device 100 of the present invention may include a solar power supply element 120. In one embodiment of the present invention, the solar power supply element 120 may be installed on an exterior housing of the portable health management device 100 and/or the display screen. Because a large amount of computing resources will be used when the analysis module 106 performs the analysis, the solar power supply element 120 may supply power through a light source, thereby extending the use time of the portable health management device 100.

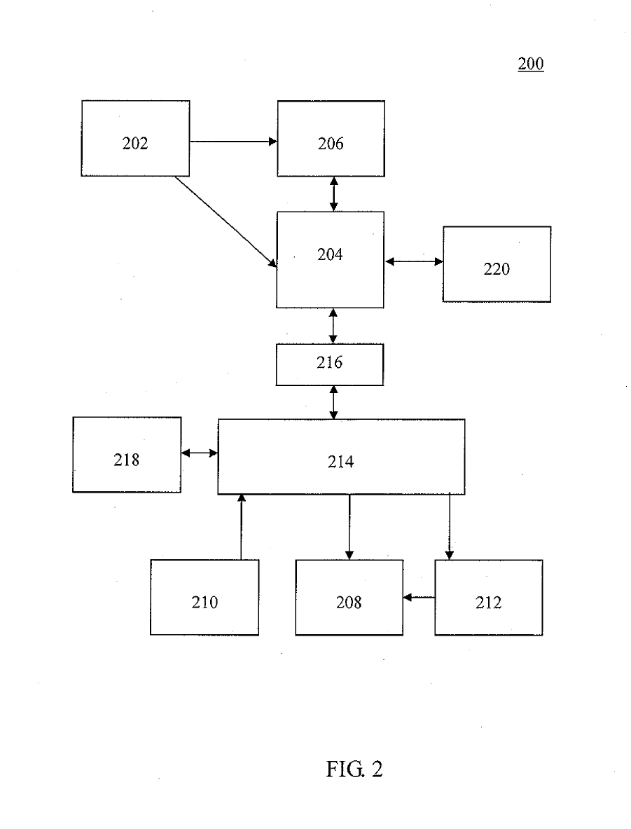

Refer to FIG. 2, which illustrates a block diagram of an exemplary health management terminal device in accordance with one embodiment of the present invention. The health management terminal device 200 of the present invention may be provided in hospitals or health center and administered by a medial staff to measure, analyze, track the variation of the biological measurements for a specific time period for the user.

As shown in FIG. 2, the health management terminal device 200 of the present invention may include a measuring unit 202 capable of measuring a user's biological measurement. For example, the measuring unit 202 may measure the user's blood pressure, blood sugar, body temperature, weight, body fat, heart beat or any other biological measurements.

The health management terminal device 200 of the present invention may include a storage unit 204. In one embodiment of the present invention, the storage unit 204 may store the biological measurements measured by the measuring unit 202, such as the user's blood pressure, blood sugar, body temperature, weight, body fat and/or heart beat. In accordance with another embodiment of the present invention, the storage unit 204 may also store at least one monitoring condition associated with the biological measurements. In accordance with one embodiment of the present invention, the monitoring condition may set to be a condition to monitor a biological measurement during a period of time. For example, when the user wishes to monitor a user's weight variation over a period of three months, time parameters, such as “three months,” “weight variation” and/or other parameters for the biological measurements may be stored in the storage unit 204. In another example, the user may wish to monitor the variation of the blood sugar levels before and after food intake over a period of half a year. In such case, time parameters, such as “six months” and “blood sugar level at noon,” and “blood sugar level at 1 pm,” as well as other parameters for the biological measurements may be stored in the storage unit 204. However, those skilled in the art should know that the monitoring condition does not necessarily include only time parameters. Instead, any parameters needed to monitor the user's biological measurements may be included.

In accordance with another embodiment of the present invention, the storage unit 204 may also store a plurality of experience data associated with the biological measurements. In accordance with one embodiment of the present invention, the experience data may be all information and knowledge about historical events and processes observed from long term clinical care that can be described by data or structured qualitative descriptions based on the characteristics of the biological measurement. For example, with respect to the characteristics of blood sugar, because blood sugar levels change before and after food intake, the associated experience data may be the variation of the blood sugar levels before and after each meal of a patience monitored on a long-term basis. Take another example of the characteristics of blood pressure. Because blood pressure may change depending on a certain time during a day and/or an activity involved, the associated experience data may be the variation of the blood pressures measured in the morning and in the afternoon of a patient with high blood pressure. In accordance with a preferred embodiment of the present invention, the experience data combines the professional knowledge of patient care attendants and the long term clinical care data. In other words, the experience data can reflect the variation of different biological measurements due to the difference in each individual's physical health condition, age and other parameters.

Because the biological measurements stored in the storage unit 204 may be personal data involving privacy and/or security issues, in one embodiment of the present invention, the storage unit 204 may also include an encryption mechanism to perform encryption on the biological measurement stored therein.

The health management terminal device 200 of the present invention may also include an authentication mechanism 216 capable of setting a plurality of user levels so that the user with different user levels may access different levels of data stored in the health management terminal device 200. For example, in one embodiment of the present invention, the health management terminal device 200 may be implemented as a weight scale placed in a hospital. Because the weight scale in the hospital may be used by many people, such as doctors, nurses, and patients, etc., in order to protect the data stored within the weight scale, the health management terminal device 200 of the present invention may set up different access rights for the users, such as doctors, nurses, and patients, etc. For example, a nurse may only access a patient's latest biological measurement of a certain kind, such as weight, whereas a doctor may access the biological measurement of a certain kind measured in the recent year, such as weight, or other associated biological measurements, such as weight and blood pressure to help facilitate the analysis.

In accordance with the present invention, the health management terminal device 200 may include a health management module 206. In one embodiment of the present invention, the health management module 206 may analyze the biological measurement obtained by the measuring unit 202 in real time based on the at least one monitoring condition stored in the storage unit 204, and generate a professional report. Because the monitoring condition considered by the health management module 206 when performing an analysis tracks the measurements obtained from the user for a long time, the professional report generated by the health management module 206 may provide a more precise analysis based on the characteristics of a particular type of biological measurements. In one embodiment of the present invention, the professional report may provide the user with a suggestion to improve the biological measurement in real time, thereby managing the user's health. For example, if the blood pressure measured for a user with a history of high blood pressure is higher than the average measurements obtained in the past three months, then the professional report may indicate that the user has an abnormal blood pressure, and suggest the user to take medication, relax, or pay attention to food intake. Doctors or medical staff may further verify the professional report so as to remind the user of the ways to improve his or her health condition. In one embodiment of the present invention, the storage unit 204 may store the professional report generated by the health management module 206 for future use. In one embodiment of the present invention, the professional report may be presented in text, graphics or the combination thereof so as to help the user understand the content of the professional report.

In accordance with the present invention, the health management terminal device 200 may include an output unit 208. In one embodiment of the present invention, the output unit 208 may include but not limited to a display screen or a speaker installed on the health management terminal device 200, an external printer, a portable drive, or any other device capable of outputting the analysis result. In one embodiment of the present invention, the output unit 208 may output the professional report in voice, graphics, text, data file, or any combination thereof.

In accordance with the present invention, the health management terminal device 200 may include a graphic processing device 212 capable of processing the graphics shown in the professional report. In one embodiment of the present invention, the graphic processing device 212 may process graphics in high resolutions so as to clearly present detailed graphics in the professional report.

The health management terminal device 200 of the present invention may include an input unit 210. In accordance with one embodiment of the present invention, the input unit 210 may include but not limited to a mouse, keyboard, speaker stalled on the health management terminal device 200, or other external input elements. In accordance with one embodiment of the present invention, the input unit 210 may allow the user to enter a monitoring condition that the health management module 206 needs to conduct the analysis, a suggestion to be provided in the professional report, or any other data associated with the user, such as a target value. For example, in one embodiment of the present invention, the input unit 210 may allow the user to enter a target value to be used as a basis for the health management module 206 to analyze the biological measurement. For example, the user may enter a target weight so that whenever the health management module 206 performs an analysis on the weight value obtained by the measuring unit 202, the target weight may be considered as one of the basis for the determination. If the weight measurement obtained from an overweight user is higher than the target value, then the professional report generated by the health management module 206 may provide the user with a suggestion to reduce his or her weight. Alternatively, if the weight measured from an underweight user is lower than the target value, then the professional report generated by the health management module 206 may provide the user with a suggestion to increase his or her weight. In one embodiment of the present invention, the storage unit 204 may store the target value entered by the user for future use.

In accordance with the present invention, the health management terminal device 200 may include a user interface 214 so that the user can control the health management terminal device 200 by voice or graphics. In one embodiment of the present invention, the user interface 214 may utilize a graphic icon or text message to guide the user to control the health management terminal device 200.

The health management terminal device 200 of the present invention may include a network communication module 218. The network communication module 218 of the present invention may allow communication with an external device or with another person through the network. For example, the user may upload the biological measurement and/or the professional report stored in the storage unit 204 to an external device through the network communication module 218. In another example, the user may conduct a video conference with another person using the network communication module 218.

The health management terminal device 200 of the present invention may include a data transmission interface 220. In one embodiment of the present invention, the data transmission interface may transmit the biological measurement to an external device or receive the biological measurement from the external device using an RS 232 or USB interface. For example, the above-mentioned portable health management device 100 may upload the biological measurement to a health management terminal device 200 through the data transmission interface 220. In one embodiment of the present invention, the storage unit 204 may also store the plurality of short-term, mid-term and/or long-term biological measurements transmitted to the health management terminal device 200 through the data transmission interface 220 for further analysis.

In one embodiment of the present invention, the health management module 206 may perform real time analysis based on the at least one monitoring condition stored in the storage unit 204 as well as the plurality of biological measurements and/or the associated experience data, which may alternatively be transmitted to the health management terminal device 220 through the data transmission interface 220 from, for example, the portable health management device 100, and generate a professional report. As such, the health management module 206 may utilize more biological data to analyze the health condition over a longer period of time, thereby generating a more precise professional report.

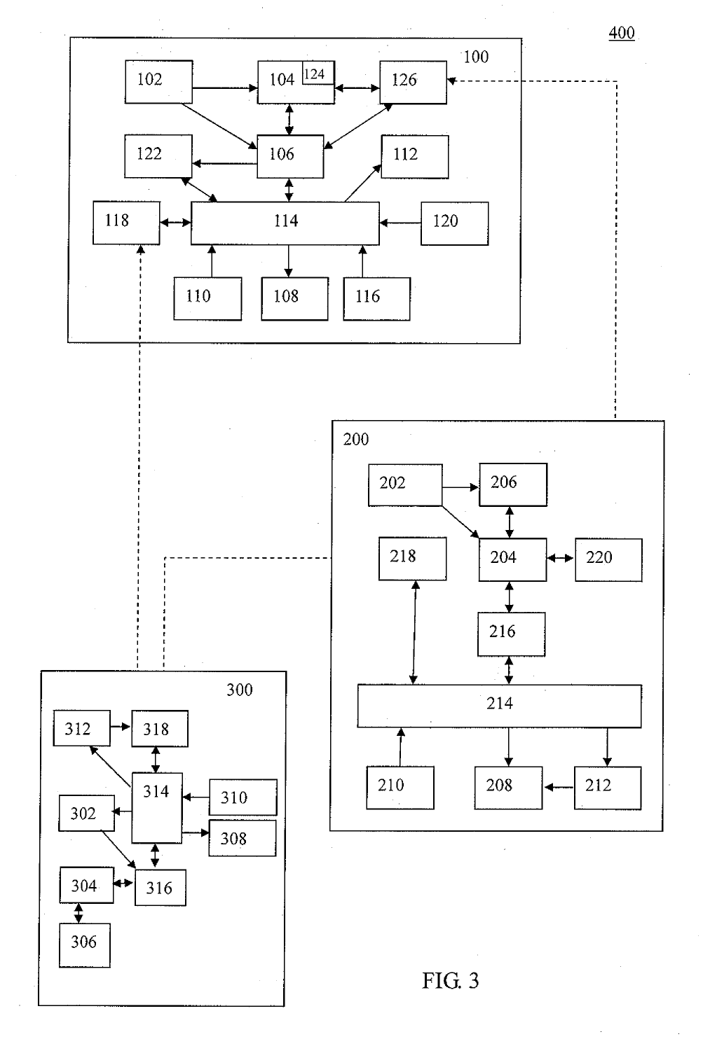

Refer to FIG. 3, which illustrates a block diagram of an exemplary health management system in accordance with one embodiment of the present invention. The health management system of the present invention may track the health data for a user over a long period of time and perform an analysis based on the tracked data. Additionally, the health management system of the present invention may allow the user to access the health report through the network. In one embodiment of the present invention, the health management system 400 may include a server 300. The server 300 of the present invention may include a registration mechanism 302 to allow the user to create an account for entering the health management system 400.

The server 300 of the present invention may also include a database 304 responsible for storing integrated health data associated with the user. In accordance with one embodiment of the present invention, the integrated health data may be merged from a particular user's biological measurements tracked over a long period of time. For example, the integrated health data may be the weight variation of a user over the past five years. Alternatively, the integrated health data may be the relational change between a user's weight and blood sugar level over the past ten years.

The health management system 400 of the present invention may include an analyzing mechanism 306. In one embodiment of the present invention, the analyzing mechanism 306 may conduct a real time analysis on the integrated health data so as to generate an integrated health report. Because the integrated health data analyzed by the analyzing mechanism 306 may be merged data from a particular user's biological measurements over a long period of time, the integrated health report generated by the analyzing mechanism 306 may provide a complete and thorough analysis and determination based on the characteristics of a particular type of biological measurement. In one embodiment of the present invention, the integrated health report may provide the user with a suggestion to improve his or her biological measurement in real time, thereby managing the user's health. For example, if the blood pressure measured from a user having a history of high blood pressure is higher than the average blood pressure measurement in the past five years, then the integrated health report may indicate that the user's blood pressure is not normal and suggest the user to take medication, relax or pay attention to food intake. In one embodiment of the present invention, the integrated health report may be presented in text, graphics or the combination thereof to help the user understand the content of the report. In one embodiment of the present invention, the database 304 may store the integrated health report for the user to access.

In accordance with the present invention, the health management system 400 may include an output device 308. In accordance with one embodiment of the present invention, the output device 308 may include but not limited to a display screen, a speaker, an external printer, a portable drive, or any other similar device capable of outputting the integrated health report. In one embodiment of the present invention, the output device 308 may output the integrated health report by voice, graphics, text, data file or any combination thereof.

In accordance with the present invention, the health management system 400 may include an input device 310. In one embodiment of the present invention, the input device 310 may include but not limited to a mouse, keyboard, microphone, or other external input device. In one embodiment of the present invention, the input device 310 may allow a user to input any parameters needed by the analyzing mechanism 306 to conduct an analysis, a suggestion to be provided in the integrated health report, or other data associated with the user, such as a target value. In one embodiment of the present invention, the input device 310 may allow the user to enter a target value to be used as a basis for the analyzing mechanism 306 to analyze the biological measurement. As such, the integrated health report provided by the analyzing mechanism 306 may further provide a suggestion to help the user achieve the target value. In one embodiment of the present invention, the database 304 may also store the target value for future analysis.

In accordance with the present invention, the health management system 400 may include a user interface 314 to allow a user to access the integrated health data, integrated health report, or the combination thereof. In one embodiment of the present invention, the user interface 314 may guide the user to control the health management system 400 through graphics or text. In one embodiment of the present invention, the user interface 314 may be a web page, allowing the user to enter the health management system 400 from a remote end.

In accordance with one embodiment of the present invention, the user may log into the health management system 400 through an account created by a registration mechanism 302 so as to access the integrated health data and integrated health report associated therewith. In accordance with one embodiment of the present invention, the health management system 400 may also include an authentication mechanism 316 so as to verify the user's access right to the system 400.

In accordance with the present invention, the health management system 400 may include a network communication module 318. The network communication module 318 may allow communication with an external device or with another person through the network. For example, the user may upload the biological measurements, the experience data, the integrated health data and/or the integrated health report stored in the storage unit 204 to an external device. In another example, the user may conduct a video conference with another person using the network communication module 318.

In accordance with the present invention, the health management system 400 may include a notification mechanism 312 to notify the user of his or her integrated health report and/or integrated health data. In one embodiment of the present invention, the notification mechanism 312 may further include a warning notification service to selectively send out a warning notification based a result shown on the integrated health report. For example, the health management system 400 may be set up so that when the integrated health report shows an abnormal health condition, a warning notification may be sent out through, for example, an email, text message, letter, or by phone.

In another embodiment of the present invention, the notification mechanism 312 may further include a target notification service so as to send out a notification based on a target value set by the user. For example, the health management system 400 may notify the user of a difference between the target value and the biological measurement shown on the integrated health report through an email, text message, letter, or by phone.

In another embodiment of the present invention, the health management system 400 may include a portable health management device 100. The portable health management device 100 may act as a user end of the health management system 400 to obtain a user's biological measurement. In one embodiment of the present invention, the portable health management device 100 may upload the short term biological measurements stored in the storage element 104 to the server 300. The portable health management device 100 may also upload the mid term and long term biological measurements stored in the storage element 104 to the server 300 depending on the capacity of the storage space. Other elements within the portable health management device 100 have already been described above and thus will not be repeated hereinafter.

In accordance with one embodiment of the present invention, the database 304 may store the plurality of biological measurements uploaded from the portable health management device 100. The plurality of biological measurements may be merged into integrated health data for the analyzing mechanism 306 to perform analysis.

In another embodiment of the present invention, the health management system 400 may include a health management terminal device 200. The health management terminal device 200 may also act as a user end of the health management system 400 to obtain a user's biological measurement. In one embodiment of the present invention, the health management terminal device 200 may upload the short term, mid term, and long term biological measurements stored in the storage unit 204 to the server 300 depending on the capacity of the storage space. Other units within the health management terminal device 200 have already been described above and thus will not be repeated hereinafter.

In one embodiment of the present invention, the database 304 may upload a plurality of biological measurements from the health management terminal device 200. The plurality of biological measurements may be merged to integrated health data for the analyzing mechanism 306 to perform analysis.

In accordance with yet another embodiment of the present invention, the health management system 400 may include both a portable health management device 100 and the health management terminal device 200. In one embodiment, the portable health management device 100 and the health management terminal device 200 may upload the plurality of biological measurements stored therein to the server 300 through the network communication modules 118 and 218, respectively. The plurality of biological measurements may be merged into integrated health data for the analyzing mechanism 306 to perform analysis.

In another embodiment of the present invention, the portable health management device 100 may first transmit the short term biological measurement stored therein to the health management terminal device 200 through the data transmission interface 126. Then, the health management terminal device 200 may upload the mid term biological measurements stored therein to the server 300 through the network communication module 218. The plurality of biological measurements may be merged into integrated health data and stored in the database 304 for the analyzing mechanism 306 to perform analysis.

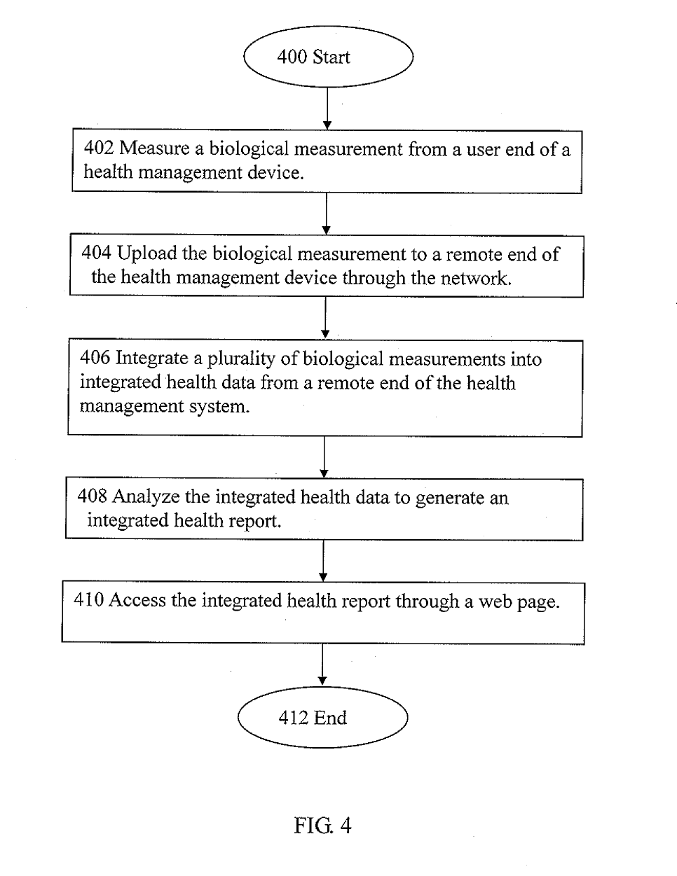

Refer to FIG. 4, which illustrates a flow chart of an exemplary health management method in accordance with one embodiment of the present invention. As shown in FIG. 4, the method starts at step 400. At step 402, the present method may measure a user's biological measurement from a user end, for example, the portable health management device 100 or the health management terminal device 200. As mentioned above, the measuring element 102 of the portable health management device 100 or the measuring unit 202 of the health management terminal device 200 may measure a user's blood pressure, blood sugar, body temperature, weight, body fat, heart beat or any other biological measurements.

Then, at step 404, for example, from the user end of the portable health management device 100 or the health management terminal device 200, the biological measurement may be uploaded to the server 300 of the health management system 400 located at a remote site through the network. Thereafter, at step 406, the remote end of the health management system 400 may integrate the plurality of biological measurements into integrated health data. As such, at step 408, the analyzing mechanism 306 at the remote health management system may analyze integrated health data in real time so as to generate an integrated health report. In accordance with one embodiment of the present invention, the integrated health data may be merged from a particular user's biological measurements kept over a long period of time. For example, the integrated health data may be a user's weight variation over the past five years. Alternatively, the integrated health data may be the relative change between a user's weight and blood sugar level over the past ten years.

At step 410, the present method may allow the user to access the integrated health report through a web page. In one embodiment of the present invention, the present method may allow the user to create an account for logging into the health management system 400 to access the health report through a registration mechanism 302 of the server 300 over the webpage. In one embodiment of the present invention, the present method may also include an authentication mechanism 316 to verify the user's access right to the system 400.

In one embodiment of the present invention, the present method may further include entering a target value so that the analyzing mechanism 306 may use such value as a basis for analyzing the integrated health data in real time. In accordance with one embodiment of the present invention, the real time analyzing step of the present method may include performing the real time analysis based on the target value and the integrated health data, and generating the integrated health report. As such, the integrated health report may provide the user with a suggestion to achieve the target value.

In accordance with one embodiment of the present invention, the present method further include a notification mechanism 312 to automatically notify the user of his or her health report and/or integrated health data. In accordance with one embodiment of the present invention, the present method further include providing a warning notification service through the notification mechanism 312 by selectively sending out a warning notification based on a result shown on the integrated health report. For example, the health management system 400 may be set up so that a warning notification may be sent through email, text message, and letter or by phone when the integrated health report shows an abnormal health condition, as explained above.

In another embodiment of the present invention, the present method may further include providing a target notification service through the notification mechanism 312 so that a notification may be sent out based on the target value set by the user. For example, the health management system 400 may notify the user of a difference between a biological measurement measured and a target value shown on the integrated health report via email, text message, letter, and/or phone, as explained above.

In accordance with one embodiment of the present invention, the present method may further include a step of outputting the integrated health report by, for example, the output device 308. In accordance with one embodiment of the present invention, the output device 308 may output the integrated health report in the form of voice, graphics, text, data file, or any combination thereof, as explained above.

In accordance with one embodiment of the present invention, the present method may further include storing the plurality of biological measurements in a remote health management system, which includes for example the database 304 of the server 300. In accordance with one embodiment of the present invention, the present method may further include storing the integrated health report in the remote health management system, which includes for example the database 304 of the server 300.

In accordance with one embodiment of the present invention, the present method may further include providing an analysis report of the biological measurements at a user end of the health management system 400, such as the portable health management device 100 or the health management terminal device 200.

While the invention has been described in conjunction with exemplary preferred embodiments, it is to be understood that many alternatives, modifications, and variations will be apparent to those skilled in the art in light of the foregoing description. Accordingly, the present invention embraces all such alternatives, modifications, and variations. All matters set forth herein or shown in the accompanying drawings are to be interpreted in an illustrative and non-limiting sense.

What is claimed is:

1. A portable health management device, said device comprising:
a measuring element measuring a biological measurement for a user;
a storage element storing a plurality of experience data associated with said biological measurement;
an analysis module analyzing said biological measurement in real time based on said plurality of experience data so as to generate an analysis result;
wherein said analysis result provides a suggestion to said user in real time to improve said biological measurement so as to manage said user's health.

2. The device of claim 1, further comprising a scheduling module placing said suggestion in a schedule so as to timely remind said user to execute said suggestion.

3. The device of claim 1, further comprising an input element inputting a target value to be a basis for said analysis module to analyze said biological measurement, wherein said analysis module generates said analysis result in real time when analyzing said biological measurement so as to provide said user with said suggestion to achieve said target value.

4. The device of claim 1, further comprising an encryption mechanism to perform encryption to said biological measurement.

5. The device of claim 1, further comprising a warning element selectively sending a warning signal based on said analysis result.

6. The device of claim 1, wherein said storage element stores said biological measurement.

7. The device of claim 1, wherein said storage element further comprises a removable memory storing a plurality of said biological measurements.

8. The device of claim 7, wherein said analysis module further performs an analysis on said plurality of said biological measurements to generate said analysis result.

9. The device of claim 1, wherein said analysis result includes a graph.

10. The device of claim 1, further comprising an output element outputting said analysis result.

11. The device of claim 10, wherein said output device outputs said analysis result in a form of voice, graph, text, data file or any combination thereof.

12. The device of claim 1, further comprising a user interface to control said device in voice or graphic form.

13. The device of claim 12, further comprising a force detection module controlling said user interface via a direction of force, wherein said force detection module asks said user to make a movement so as to determine a health condition of said user by a characteristic of said movement.

14. The device of claim 1, further comprising a network communication module to communicate with an external device or another person through a network.

15. The device of claim 1, further comprising a solar power supply element to extend a use time of said device by obtaining power through a light.

16. The device of claim 1, wherein said storage element further stores said analysis result.

17. The device of claim 1, further comprising a data transmission interface to transmit said biological measurement to an external device.

18. A health management terminal device, said device comprising:
a measuring unit measuring a biological measurement for a user;
a storage unit storing at least one monitoring condition associated with said biological measurement;
a health management module analyzing said biological measurement in real time based on said at least one monitoring condition so as to generate a professional report,
wherein said professional report provides a suggestion to said user in real time to improve said biological measurement so as to manage said user's health.

19. The device of claim 18, further comprising a data transmission interface to transmit and receive said biological measurement to and from an external device.

20. The device of claim 19, wherein said health management module analyzes said biological measurement in real time based on said at least one monitoring condition and through a plurality of biological measurements transmitted to said device through said data transmission interface so as to generate said professional report.

21. The device of claim 19, wherein said storage unit stores said plurality of biological measurements transmitted to said device through said data transmission interface.

22. The device of claim 18, wherein said storage unit stores said professional report.

23. The device of claim 18, further comprising a graphic processing device to process a graphic shown in said professional report.

24. The device of claim 18, further comprising an authentication mechanism to setup a plurality of user levels so as to provide different accesses to said device to users associated with different user levels.

25. The device of claim 18, further comprising an output unit outputting said professional report.

26. The device of claim 25, wherein said output unit outputs said professional report in a form of voice, graph, text, data file or any combination thereof.

27. The device of claim 18, further comprising a user interface to control said device in voice or graphic form.

28. The device of claim 18, further comprising a network communication module to communicate with an external device or another person through a network.

29. The device of claim 18, further comprising an input unit inputting said at least one monitoring condition, said suggestion or other data associated with said user.

30. A health management system, said system comprising:
a server, said server comprising:
a registration mechanism establishing an account for a user to enter said system;
a database storing an integrated health data associated with said user;
an analyzing mechanism analyzing said integrated health data in real time to generate an integrated health report,
wherein said integrated health data integrates a plurality of biological measurements transmitted to said server from another device, and
wherein said user enters said system using said account to access said integrated health data, said integrated health report or a combination thereof associated with said user.

31. The system of claim 30, wherein said server further comprises an authentication mechanism to authenticate a right to access said system by said user.

32. The system of claim 30, wherein said server further comprises a network communication interface to communicate with an external device or another person through a network.

33. The system of claim 30, wherein said another device can be a portable health management device to upload said plurality of biological measurements through said network communication interface.

34. The system of claim 30, wherein said another device can be a health management terminal device to upload said plurality of biological measurements through said network communication interface.

35. The system of claim 28, wherein said server further comprises a user interface allowing a user to access said integrated health data, said integrated health report or a combination thereof associated with said user.

36. The system of claim 35, wherein said user interface can be a web page allowing said user to remotely enter said system.

37. The system of claim 30, wherein said server further comprises a notification mechanism to notify said user of said integrated health report.

38. The system of claim 37, wherein said notification mechanism further comprises a warning notification service selectively sending out a warning notification based on a result shown said integrated health report.

39. The system of claim 37, wherein said notification mechanism further comprises a target notification service to send out a notification based on a target value set by said user.

40. The system of claim 30, wherein said database stores said plurality of biological measurements uploaded by said another device.

41. The system of claim 30, wherein said database stores said integrated health report.

42. The system of claim 30, wherein said server further comprises an input device inputting a target value for said user.

43. The system of claim 30, wherein said integrated health report provides said user a suggestion to achieve said target value.

44. The system of claim 30, wherein said integrated health report provides said user with a suggestion to improve said integrated health data.

45. The system of claim 30, wherein said server further comprises an output device outputting said integrated health report.

46. A health management system, said system comprising:
a health management user-end device, said device comprising a measuring element measuring a biological measurement for a user;
a network communication interface allowing said health management user-end device to upload a plurality of said biological measurements to a server,
said server comprising:
an analyzing mechanism analyzing said plurality of biological measurements in real time to generate an integrated health report; and
a user interface allowing said user to access said integrated health report.

47. The system of claim 46, wherein said server further comprises a registration mechanism allowing said user to establish an account to enter said system.

48. The system of claim 46, wherein said server further comprises an output device outputting said integrated health report.

49. The system of claim 46, wherein said server further comprises an authentication mechanism to authenticate a right to access said system by said user.

50. The system of claim 46, wherein said health management user-end device is capable of uploading a plurality of short-term, mid-term, and/or long-term biological measurements through said network communication interface.

51. The system of claim 46, wherein said user interface includes a web page allowing said user to remotely enter said system.

52. The system of claim 46, wherein said server further comprises a notification mechanism to notify said user of said integrated health report.

53. The system of claim 52, wherein said notification mechanism further comprises a warning notification service to selectively send out a warning notification based on a result shown on said integrated health report.

54. The system of claim 52, wherein said notification mechanism further comprises a target notification service to send out a notification based on a target value set by said user.

55. The system of claim 46, wherein said server further comprises a database to store said plurality of biological measurements.

56. The system of claim 55, wherein said database stores said integrated health report.

57. The system of claim 46, further comprising an input device to input a target value for said user.

58. The system of claim 57, wherein said integrated health report provides said user with a suggestion to achieve said target value.

59. The system of claim 55, wherein said database integrates said plurality of biological measurements into integrated health data.

60. The system of claim 46, further comprising an output device to output said integrated health report.

61. A method for health management, said method comprising:
measuring a biological measurement at a user end of a health management system;
uploading said biological measurement to a remote end of said health management system through a network;
integrating a plurality of said biological measurements into integrated health data at said remote end of said health management system; and
analyzing said integrated health data in real time to generate an integrated health report.

62. The method of claim 61, further comprising using an account to enter said remote end of said health management system through a web page to access said integrated health report.

63. The method of claim 61, further comprising automatically notifying a user of said integrated health report.

64. The method of claim 61, further comprising selectively sending out a warning notification based on a result shown on said integrated health report.

65. The method of claim 61, further comprising sending out a notification based on a target value set by a user.

66. The method of claim 61, further comprising authenticating a right to access said remote end of said health management system by a user.

67. The method of claim 61, further comprising outputting said integrated health report.

68. The method of claim 61, further comprising storing said plurality of biological measurements in said remote end of said health management system.

69. The method of claim 61, further comprising storing said integrated health report in said remote health management system.

70. The method of claim 61, further comprising providing an analysis report at said user end of said health management system.

71. The method of claim 61, further comprising entering a target value as a basis for analyzing said integrated health data in real time.

72. The method of claim 71, further comprising performing real time analysis based on said target value and said integrated health data to generate said integrated health report, wherein said integrated health report provides said user with a suggestion to achieve said target value.
